(19) United States
(12) Patent Application Publication
(10) Pub. No.: US 2007/0112057 A1
(43) Pub. Date: May 17, 2007

(54) Polymorphic forms of tegaserod maleate

(76) Inventors: Santiago Ini, Haifa (IL); Tamas Koltai, Netanya (IL); Michael Pinchasov, Dover, NJ (US); Sofia Gorohovsky, Kiriat Gat (IL); Gustavo Frenkel, Beer Sheva (IL)

Correspondence Address: KENYON & KENYON LLP, ONE BROADWAY, NEW YORK, NY, 10004, US

(21) Appl. No.: 11/473,582

(22) Filed: Jun. 22, 2006

Related U.S. Application Data
(60) Provisional application No. 60/693,301, filed on Jun. 22, 2005.
(60) Provisional application No. 60/704,048, filed on Jul. 28, 2005.
(60) Provisional application No. 60/721,729, filed on Sep. 28, 2005.
(60) Provisional application No. 60/729,258, filed on Oct. 20, 2005.
(60) Provisional application No. 60/773,066, filed on Feb. 13, 2006.
(60) Provisional application No. 60/792,811, filed on Apr. 17, 2006.

(51) Int. Cl. A61K 31/405; C07D 209/18

(52) U.S. Cl. 514/419; 548/495

(57) ABSTRACT

Provided are crystalline forms of tegaserod maleate and processes for the preparation thereof.

### CROSS-REFERENCED TO RELATED APPLICATIONS

This application claims the benefit of the U.S. Provisional Application No. 60/693,301, filed Jun. 22, 2005; 60/704,048, filed Jul. 28, 2005; 60/721,729, filed Sep. 28, 2005; 60/729,258, filed Oct. 20, 2005; 60/773,066, filed Feb. 13, 2006; and 60/792,811, filed Apr. 17, 2006. The contents of which are incorporated herein by reference.

### FIELD OF THE INVENTION

The invention encompasses tegaserod maleate crystalline forms and processes for preparing tegaserod maleate crystalline forms.

### BACKGROUND OF THE INVENTION

Tegaserod maleate is an aminoguanidine indole 5HT4 agonist for the treatment of irritable bowel syndrome (IBS). Tegaserod maleate is also known as 3-(5-methoxy-1H-indole-3-ylmethylene)-N-pentylcarbazimidamide hydrogen maleate, and has the following structure:

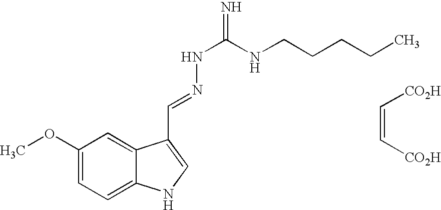

Tegaserod maleate is a white to off-white powder slightly soluble in ethanol and very slightly soluble in water. Physician's Desk Reference, 57^{th} ed., p. 2339. The marketed polymorphic form of tegaserod maleate (ZELNORM) is listed in IPCOM000021161D and designated tegaserod maleate Form A.

The invention relates to the solid state physical properties of tegaserod maleate. These properties may be influenced by controlling the conditions under which tegaserod maleate is obtained in solid form. Solid state physical properties include, for example, the flowability of the milled solid. Flowability affects the ease with which the material is handled during processing into a pharmaceutical product. When particles of the powdered compound do not flow past each other easily, a formulation specialist must take that fact into account in developing a tablet or capsule formulation, which may necessitate the use of glidants such as colloidal silicon dioxide, talc, starch or tribasic calcium phosphate.

Another important solid state property of a pharmaceutical compound is its rate of dissolution in aqueous fluid. The rate of dissolution of an active ingredient in a patient's stomach fluid may have therapeutic consequences since it imposes an upper limit on the rate at which an orally-administered active ingredient may reach the patient's bloodstream. The rate of dissolution is also a consideration in formulating syrups, elixirs and other liquid medicaments. The solid state form of a compound may also affect its behavior on compaction and its storage stability.

These practical physical characteristics are influenced by the conformation and orientation of molecules in the unit cell, which defines a particular form of a substance.

Tegaserod maleate is disclosed in U.S. Pat. No. 5,510,353 (Example 13) and the equivalent EP 0 505 322. The '353 patent discloses the preparation of tegaserod base by reacting indole-3-carbaldehyde and aminoguanidine in a protic solvent in the presence of inorganic or organic acid (Example 2a describes the reaction in methanol and hydrochloric acid). Tegaserod maleate disclosed in the '353 patent is reported to have a melting point of 190° C. (Table 1, Example 13).

The literature (Buchheit K. H., et al., J. Med. Chem., 1995, 38, 2331) describes a general method for the condensation of aminoguanidines with indole-3-carbaldehydes in methanol in the presence of HCl (pH 3-4). The product obtained after solvent evaporation may be converted to its hydrochloride salt by treatment of the methanolic solution with diethylether/HCl followed by recrystallization from methanol/diethylether. Tegaserod base prepared according to this general method is characterized solely by a melting point of 155 ° C. (table 3 compound 5b). Tegaserod maleate characterization was done by ¹H and ¹³C-NMR according to the literature (Jing J. et. al., Guangdong Weiliang Yuansu Kexue, 2002, 9/2, 51).

WO 05/014544 discloses Form A, or “Modification A,” with an X-ray diffraction pattern having peaks at about 5.4, 5.9, 6.4, 10.8, 16.2, 19.3, 21.7 and 26.8±0.3 degrees two theta. The reference also discloses a crystalline form of tegaserod maleate, “Modification B,” having peaks at about 7.7, 8.7, 21.6, 25.1 and 27.0±0.3 degrees two theta.

Chinese patent No. CN 1176077 C, discloses X-ray diffractograms of two tegaserod maleate crystalline forms, therein designated Form S and Form W.

WO 04/085393 discloses four crystalline forms of tegaserod maleate, therein denominated crystal forms I, II, III and IV.

The discovery of new forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. There is a need in the art for additional forms of tegaserod maleate and/or processes for their preparation.

### SUMMARY OF THE INVENTION

The present invention provides a crystalline form of tegaserod maleate characterized by X-ray powder diffraction peaks at about 6.6, 7.9, 8.9, 19.7 and 27.2±0.2 degrees two theta, wherein the crystalline form is substantially free of a peak at about 10.3±0.2 degrees two theta.

Another embodiment of the invention encompasses methods for preparing crystalline tegaserod maleate characterized by an X-ray diffraction pattern with peaks at about 5.4, 6.0, 6.6 and 10.8±0.2 degrees two theta, comprising spray drying a solution of tegaserod maleate.

In another embodiment, the invention encompasses a process for preparing crystalline tegaserod maleate characterized by an X-ray diffraction pattern with peaks at about 5.4, 6.0, 6.6 and 10.8±0.2 degrees two theta, comprising grinding a mixture of tegaserod hemi-maleate hemihydrate with maleic acid.

In another embodiment, the invention encompasses a process for preparing crystalline tegaserod maleate characterized by an X-ray diffraction pattern with peaks at about 5.4, 6.0, 6.6 and 10.8±0.2 degrees two theta comprising combining a slurry of tegaserod hemi-maleate hemihydrate in a solvent selected from the group consisting of: ethyl acetate, diisopropyl ether [DIPE], 2-methyl-THF, water, acetonitrile, n-butanol, sec-butanol, methyl isobutyl ketone, toluene, heptane, MEK and mixtures thereof, with maleic acid to obtain a mixture, maintaining the mixture to obtain a solid and recovering the obtained crystal form.

In another embodiment, the invention encompasses a process for preparing crystalline tegaserod maleate characterized by an X-ray diffraction pattern with peaks at about 15.7, 16.9, 17.2, 24.1, 24.6 and 25.2±0.2 degrees two theta comprising combining a slurry of tegaserod hemi-maleate hemihydrate in n-propanol, with maleic acid to obtain a mixture, maintaining the mixture to obtain a solid and recovering the obtained crystal form.

In another embodiment, the invention encompasses a process for preparing crystalline tegaserod maleate characterized by an X-ray diffraction pattern with peaks at about 15.7, 16.9, 17.2, 24.1, 24.6 and 25.2±0.2 degrees two theta comprising combining tegaserod maleate crystalline form characterized by an X-ray diffraction pattern with peaks at about 8.7, 15.6, 16.0, 22.2 and 25.3±0.2 degrees two theta with ethyl acetate and n-propanol to obtain a mixture, heating the mixture to a temperature of about 100° C. to reflux, cooling the mixture to about room temperature or less, and recovering the obtained crystal form.

In another embodiment, the invention encompasses a process for crystallizing tegaserod maleate characterized by an X-ray diffraction pattern with peaks at about 15.7, 16.9, 17.2, 24.1, 24.6 and 25.2±0.2 degrees two theta from a solution of tegaserod maleate, ethyl acetate and n-propanol.

In another embodiment, the invention encompasses a process for preparing crystalline tegaserod maleate characterized by an X-ray diffraction pattern with peaks at about 6.6, 7.9, 8.9, 19.7, 21.8, 23.0, 23.9, 25.3 and 27.2±0.2 degrees two-theta comprising combining a slurry of tegaserod hemi-maleate hemihydrate in methanol, with maleic acid to obtain a mixture, maintaining the mixture to obtain a solid and recovering the obtained crystal form.

In another embodiment, the invention encompasses a process for crystallizing tegaserod maleate characterized by an X-ray diffraction pattern with peaks at about 6.6, 7.9, 8.9, 19.7, 21.8, 23.0, 23.9, 25.3 and 27.2±0.2 degrees two-theta from a solution of tegaserod hemi-maleate hemihydrate, maleic acid and methanol

### BRIEF DESCRIPTION OF THE FIGURES

FIG. 1 is an X-ray powder diffraction pattern for crystalline tegaserod maleate Form A.

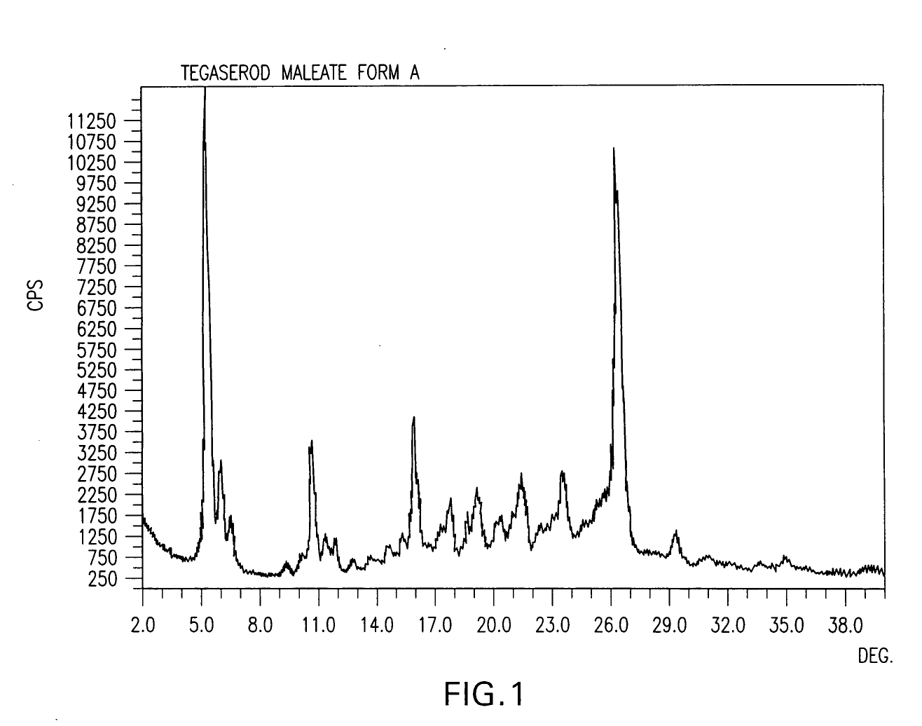

FIG. 2 is an X-ray powder diffraction pattern for crystalline tegaserod maleate Form B

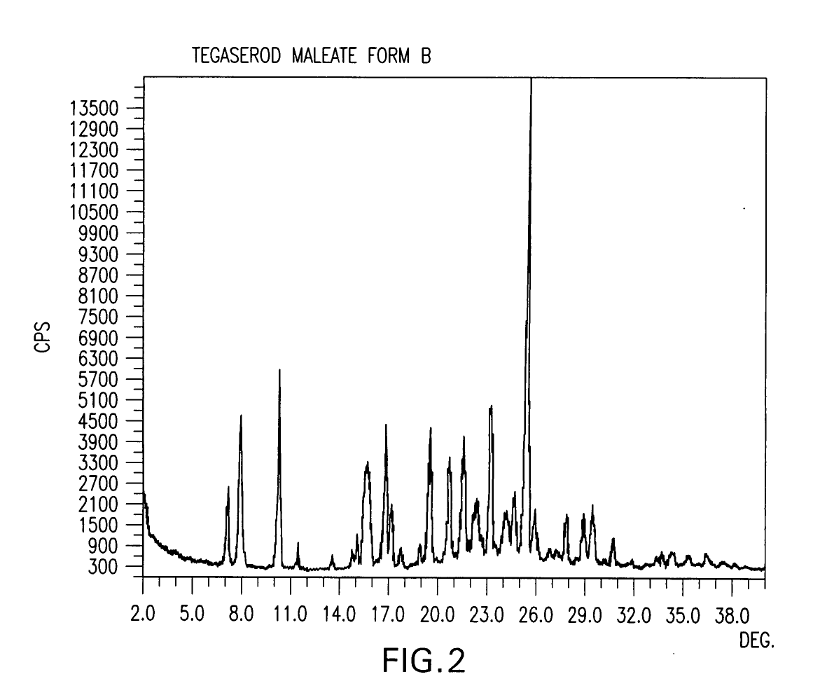

FIG. 3 is an X-ray powder diffraction pattern for crystalline tegaserod maleate Form B1.

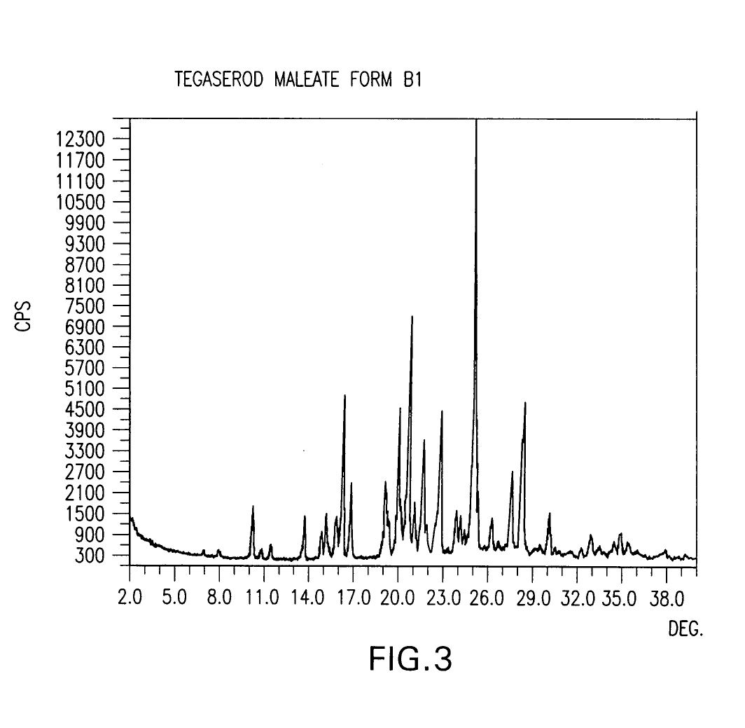

FIG. 4 is an X-ray powder diffraction pattern for crystalline tegaserod maleate Form B2.

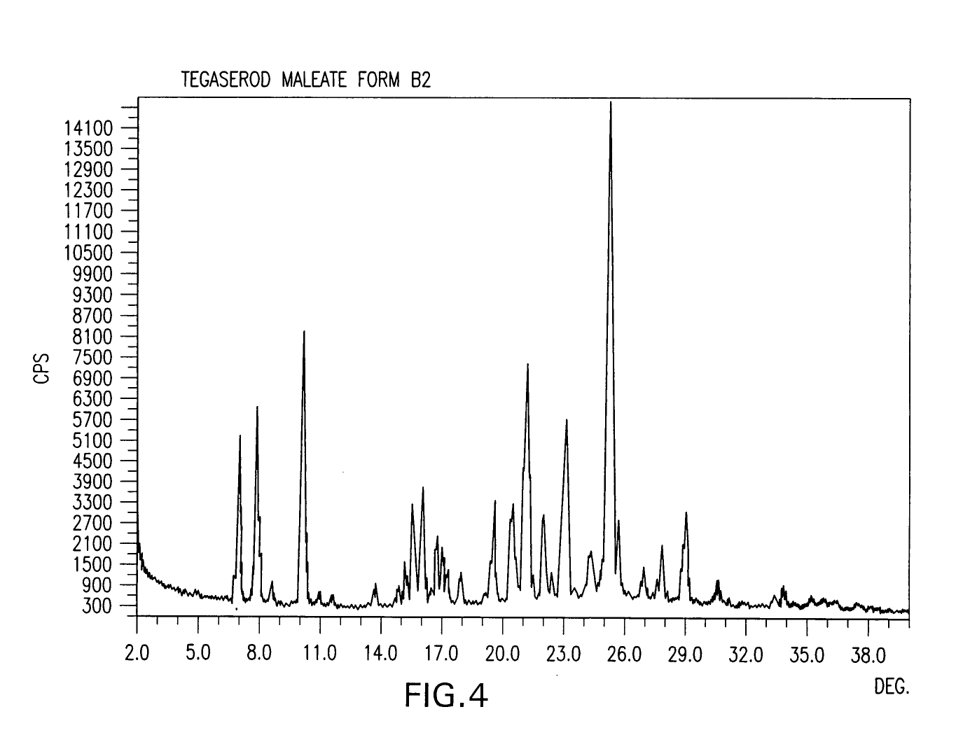

FIG. 5 is an X-ray powder diffraction pattern for crystalline tegaserod maleate Form B3.

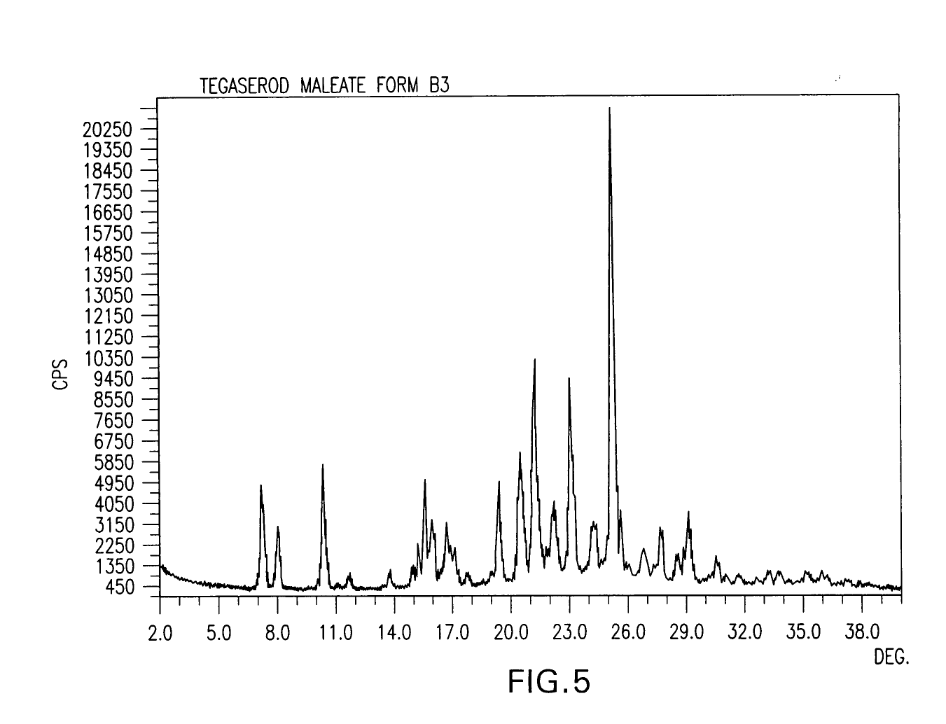

FIG. 6 is an X-ray powder diffraction pattern for crystalline tegaserod maleate Form C.

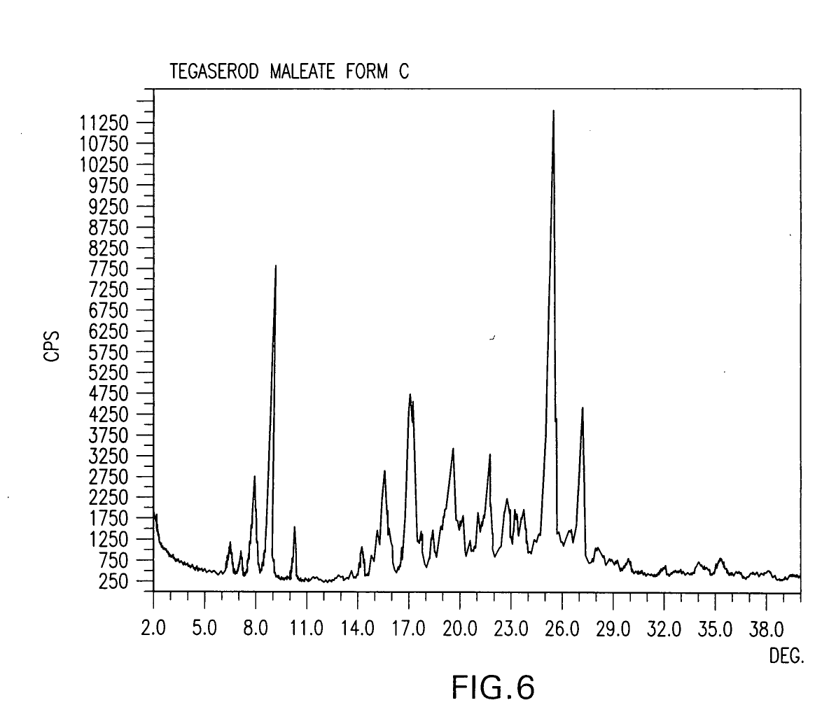

FIG. 7 is an X-ray powder diffraction pattern for crystalline tegaserod maleate Form M.

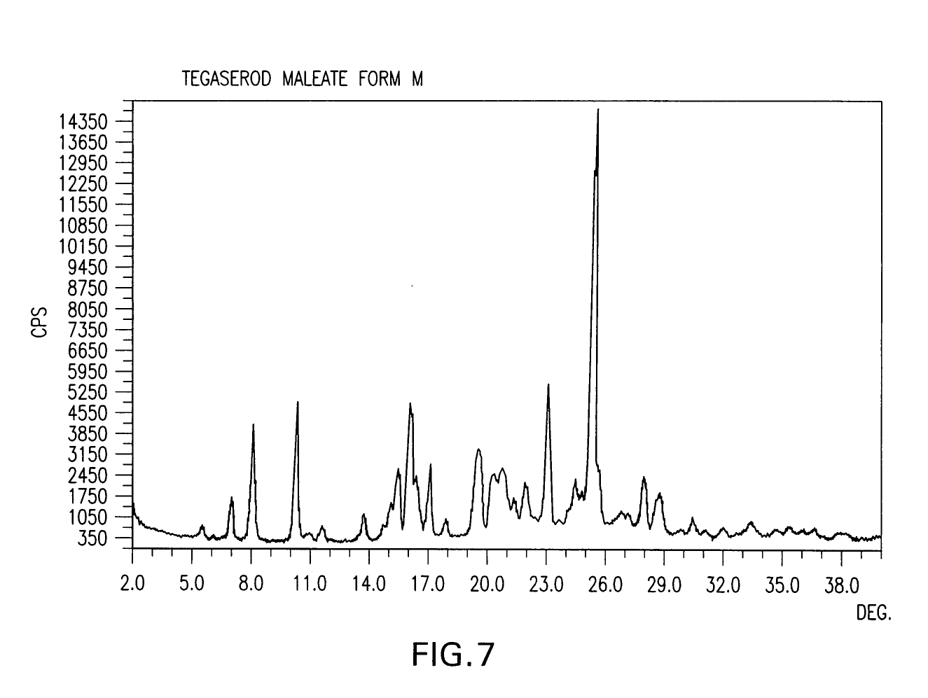

FIG. 8 is an X-ray powder diffraction pattern for crystalline tegaserod maleate Form Z.

### DETAILED DESCRIPTION OF THE INVENTION

As used herein, the term “non-hygroscopic” refers to a compound that does not absorb more than 0.2% of water at 80% humidity, at a temperature of 25° C. for 24 hours, as described in Pharmeuropa, Vol. 4, No. 3, September 1992.

The present invention provides a crystalline form of tegaserod maleate characterized by X-ray powder diffraction peaks at about 6.6, 7.9, 8.9, 19.7 and 27.2±0.2 degrees two-theta, wherein the crystalline form is substantially free of a peak at about 10.3±0.2 degrees two theta. This form is denominated Form Z. Form Z may further be characterized by X-ray powder diffraction peaks at about 21.8, 23.0, 23.9 and 25.3±0.2 degrees two-theta.

Preferably, the peak at about 10.3±0.2 degrees two theta is absent wherein the analysis is done at a scan rate slow enough, according to the common knowledge of the skilled in the art. The scan rate used may vary from instrument to instrument, and sample preparation.

Preferably, Form Z contains less than about 5% of any other crystalline form of tegaserod maleate by weight, more preferably less than about 1% by weight.

Preferably, Form Z form is free of detectable peaks at about 7.0, 10.3, 13.7, 20.7 and 23.2±0.2 degrees two theta. More preferably, the peaks at about 7.0, 10.3, 13.7, 20.7 and 23.2±0.2 degrees two theta are absent wherein the analysis is done at a scan rate slow enough, according to the common knowledge of the skilled in the art. The scan rate used may vary from instrument to instrument, and sample preparation.

Form Z was found to be anhydrous.

Form Z has a weight loss of about 0.1% by weight at the range of about 25° C. to about 200° C., as measured by TGA.

Form Z was found to be non hygroscopic when tested for water absorption at room temperature for 37 days under 80% relative humidity.

The present invention further provides a process for preparing Form Z comprising drying crystalline tegaserod maleate characterized by an X-ray diffraction pattern with peaks at about 6.6, 7.9, 8.9, 19.7, 21.8, 23.0, 23.9, 25.3 and 27.2±0.2 degrees two-theta (Form C), contaminated with other polymorphic forms at a temperature of about 110° C. for at least about 2 hours.

Form C may be obtained by any method known in the art, such as described in WO 04/085393.

The present invention further provides methods for preparing crystalline tegaserod maleate characterized by an X-ray diffraction pattern with peaks at about 5.4, 6.0, 6.6 and 10.8±0.2 degrees two theta (Form A), comprising spray drying a solution of tegaserod maleate.

The term “spray drying” broadly refers to processes involving breaking up liquid mixtures into small droplets (atomization) and rapidly removing solvent from the mixture. In a typical spray drying apparatus, a strong driving force evaporates the solvent from the droplets, which may be provided by providing a drying gas. Spray drying processes and equipment are described in Perry's Chemical Engineer's Handbook, pgs. 20-54 to 20-57 (Sixth Edition 1984).

By way of non-limiting example only, the typical spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream from the drying chamber. Examples of such apparatuses include Niro Models PSD-1, PSD-2 and PSD-4 (Niro A/S, Soeborg, Denmark). Typically, the product collection means includes a cyclone connected to the drying apparatus. In the cyclone, the particles produced during spray drying are separated from the drying gas and evaporated solvent, allowing the particles to be collected. A filter may also be used to separate and collect the particles produced by spray drying. The process of the invention is not limited to the use of such drying apparatuses as described above.

Spray drying may be performed in a conventional manner in the processes of the present invention (see, e.g., Remington: The Science and Practice of Pharmacy, 19th Ed., vol. II, pg. 1627, herein incorporated by reference). The drying gas used in the invention may be any suitable gas, although inert gases such as nitrogen, nitrogen-enriched air, and argon are preferred. Nitrogen gas is a particularly preferred drying gas for use in the process of the invention. The tegaserod maleate product produced by spray drying may be recovered by techniques commonly used in the art, such as using a cyclone or a filter.

Crystalline Form A is obtained by spray drying a solution of tegaserod maleate at a wide inlet/outlet temperature range.

In one embodiment, Form A is obtained by spray drying a solution of tegaserod maleate in a solvent selected from the group consisting of: amines, amides, ketones and mixtures thereof with C₁-C₈ alcohols or water.

Preferably, the solvent is selected from the group consisting of: N-methyl-2-pyrrolidine or mixture thereof with methanol, N,N-dimethylformamide, and a mixture of acetone and water.

Preferably, wherein a mixture of N-methyl-2-pyrrolidine with methanol is used as a solvent, the N-methyl-2-pyrrolidine used is in a ratio of about 1:1 to about 1:4 by volume of methanol used.

Preferably, wherein a mixture of acetone and water is used as a solvent, the acetone used is in a ratio of about 4:1 of water used.

Preferably, the solution is spray-dried at an inlet temperature of from about 30° C. to about 200° C., more preferably from about 50° C. to about 200° C., and most preferably from about 50° C. to about 150° C. The outlet temperature is below the inlet temperature

Form A in a mixture with other forms may also be obtained by spray drying a solution of tegaserod maleate in ethanol. The solution preferably contains of about 10% to about 40%, more preferably about 25% water by volume.

In one embodiment, the invention encompasses a process for obtaining a mixture of Form A and a crystalline form characterized by an X-ray diffraction pattern with peaks at about 15.6, 16.0, 22.5, 25.5 and 29.3±0.2 degrees two theta (Form B3) by spray drying a solution of tegaserod maleate in water and ethanol at an inlet temperature of from about 80° C. to about 120° C.

Preferably, the solution is spray-dried at an inlet temperature of from about 90° C. to about 110° C., and more preferably about 100° C.

In another embodiment, the invention encompasses a process for obtaining a mixture of Form A and a crystalline form characterized by an X-ray diffraction pattern with peaks at about 8.7, 15.6, 16.0, 22.2 and 25.3±0.2 degrees two theta (Form B2) by spray drying a solution of tegaserod maleate in water and ethanol at an inlet temperature of from about 30° C. to about 70° C.

Preferably, the solution is spray-dried at an inlet temperature of from about 40° C. to about 60° C., and more preferably about 50° C.

In another embodiment, the invention encompasses a process for preparing tegaserod maleate Form A comprising grinding a mixture of tegaserod hemi-maleate hemihydrate with maleic acid.

Preferably, the tegaserod hemi-maleate hemihydrate is present in a ratio of about 1:1 weight /volume of maleic acid.

Form A may then be recovered by any method known in the art.

In another embodiment, the invention encompasses a process for preparing tegaserod maleate Form A comprising combining a slurry of tegaserod hemi-maleate hemihydrate in a solvent selected from the group consisting of: ethyl acetate, diisopropyl ether [DIPE], 2-methyl-THF, water, acetonitrile, n-butanol, sec-butanol, methyl isobutyl ketone, toluene, heptane, MEK or a mixture thereof, with maleic acid to obtain a mixture, maintaining the mixture to obtain a solid and recovering Form A.

Before combining the slurry with maleic acid, the slurry may be heated to a temperature of from about room temperature to about 70° C., more preferably to a temperature of from about 60° C. to about 65° C. If the slurry is heated, the process may further comprise cooling the mixture. Preferably, the mixture is cooled to room temperature.

Preferably, the maleic acid is added as a solution with the same solvent used to form the slurry.

Optionally, the mixture is treated with an ultrasound probe (sonicator).

Preferably, the mixture is maintained while stirring for about 5 minutes to about 15 hours.

Form A may then be recovered by any method known in the art.

In another embodiment, the invention encompasses a process for preparing crystalline tegaserod maleate characterized by an X-ray diffraction pattern with peaks at about 15.7, 16.9, 17.2, 24.1, 24.6 and 25.2±0.2 degrees two theta (Form B) comprising combining a slurry of tegaserod hemi-maleate hemihydrate in n-propanol, with maleic acid to obtain a mixture, maintaining the mixture to obtain a solid and recovering Form B.

Before combining the slurry with maleic acid, the slurry may be heated to a temperature of from about room temperature to about 70° C., more preferably to a temperature of from about 60° C. to about 65° C. If the slurry is heated, the process may further comprise cooling the mixture. Preferably, the mixture is cooled to room temperature.

Preferably, the maleic acid is added as a solution with the same solvent used to form the slurry.

Preferably, the mixture is maintained while stirring for about 5 minutes to about 15 hours.

Form B may then be recovered by any method known in the art.

In another embodiment, the invention encompasses a process for preparing tegaserod maleate Form B comprising combining tegaserod maleate Form B2 with ethyl acetate and n-propanol to obtain a mixture, heating the mixture to a temperature of about 100° C. to reflux, cooling the mixture to about room temperature or less, and recovering the obtained tegaserod maleate Form B.

Preferably, the mixture is heated to a temperature of about 100° C.

Preferably, after the heating, the mixture is maintained, while stirring, preferably for at least 0.5 hour.

Preferably, the mixture is cooled to a temperature of about 10° C.

In another embodiment, the invention encompasses a process for crystallizing Form B from a solution of tegaserod maleate, ethyl acetate and n-propanol.

Preferably, the ethyl acetate used is in a ratio of about 1:1 to about 1:3 by volume to n-propanol used.

Preferably, the solution is maintained, while stirring, at room temperature, for about 7 hours, to obtain Form B.

Tegaserod maleate Form B may then be recovered by any method known in art.

In another embodiment, the invention encompasses a process for preparing crystalline tegaserod maleate characterized by an X-ray Diffraction pattern having peaks at about 10.3, 16.1, 16.5, 17.1, 20.3, 22.0 and 25.3±0.2 degrees two theta (Form B1) comprising combining a slurry of tegaserod hemi-maleate hemihydrate in iso-propyl alcohol (IPA), with maleic acid to obtain a mixture, maintaining the mixture to obtain a solid and recovering Form B1.

Before combining the slurry with maleic acid, the slurry may be heated to a temperature of from about room temperature to about 70° C., more preferably to a temperature of from about 60° C. to about 65° C. If the-slurry is heated, the process may further comprise cooling the mixture. Preferably, the mixture is cooled to room temperature.

Preferably, the maleic acid is added as a solution with IPA.

Preferably, the mixture is maintained while stirring for about 5 minutes to about 15 hours, more preferably for about 3 hours.

Form B1 may then be recovered by any method known in the art.

In another embodiment, the invention encompasses a process for preparing Form B2 comprising combining a slurry of tegaserod hemi-maleate hemihydrate in ethanol/water, with maleic acid to obtain a mixture, maintaining the mixture to obtain a solid and recovering Form B2.

Before combining the slurry with maleic acid, the slurry may be heated to a temperature of from about room temperature to about 70° C., more preferably to a temperature of from about 60° C. to about 65° C. If the slurry is heated, the process may further comprise cooling the mixture. Preferably, the mixture is cooled to room temperature.

Preferably, the ethanol used, is in a ratio of about 1:1, 8:2 or 7:3 by volume to water used.

Preferably, the maleic acid is added as a solution with ethanol/water.

Preferably, the mixture is maintained while stirring for about 5 minutes to about 15 hours, more preferably for about 3 hours.

Form B2 may then be recovered by any method known in the art.

In another embodiment, the invention encompasses a process for preparing Form B3 comprising combining a slurry of tegaserod hemi-maleate hemihydrate in ethanol, with maleic acid to obtain a mixture, maintaining the mixture to obtain a solid and recovering Form B3.

Before combining the slurry with maleic acid, the slurry may be heated to a temperature of from about room temperature to about 70° C., more preferably to a temperature of from about 60° C. to about 65° C. If the slurry is heated, the process may further comprise cooling the mixture. Preferably, the mixture is cooled to room temperature.

Preferably, the maleic acid is added as a solution with ethanol.

Preferably, the mixture is maintained while stirring for about 5 minutes to about 15 hours, more preferably for about 3 hours.

Form B3 may then be recovered by any method known in the art.

In another embodiment, the invention encompasses a process for preparing tegaserod maleate Form M comprising combining a slurry of tegaserod hemi-maleate hemihydrate in acetone, with maleic acid to obtain a mixture, maintaining the mixture to obtain a solid and recovering Form M.

Before combining the slurry with maleic acid, the slurry may be heated to a temperature of from about room temperature to about 70° C., more preferably to a temperature of from about 60° C. to about 65° C. If the slurry is heated, the process may further comprise cooling the mixture. Preferably, the mixture is cooled to room temperature.

Preferably, the maleic acid is added as a solution with acetone.

Preferably, the mixture is maintained while stirring for about 5 minutes to about 15 hours, more preferably for about 3 hours.

Form M may then be recovered by any method known in the art.

In another embodiment, the invention encompasses a process for crystallizing Form M from a solution of tegaserod maleate and a mixture of ethyl acetate and acetonitrile.

Preferably, the ethyl acetate used is in a ratio of about 1:3 by volume to acetonitrile used.

Preferably, the solution is maintained, while stirring, at room temperature, for about 1.5 hours, to obtain Form M.

Tegaserod maleate Form M may then be recovered by any method known in art.

In another embodiment, the invention encompasses a process for preparing tegaserod maleate Form C comprising combining a slurry of tegaserod hemi-maleate hemihydrate in methanol, with maleic acid to obtain a mixture, maintaining the mixture to obtain a solid and recovering Form C.

Before combining the slurry with maleic acid, the slurry may be heated to a temperature of from about room temperature to about 70° C., more preferably to a temperature of from about 60° C. to about 65° C. If the slurry is heated, the process may further comprise cooling the mixture. Preferably, the mixture is cooled to room temperature.

Preferably, the maleic acid is added as a solution with acetone.

Preferably, the mixture is maintained while stirring for about 5 minutes to about 15 hours, more preferably for about 3 hours.

Form C may then be recovered by any method known in the art.

In another embodiment, the invention encompasses a process for crystallizing Form C from a solution of tegaserod hemi-maleate hemihydrate, maleic acid and methanol.

Preferably, the maleic acid used is in a ratio of about 1:10 to about 1:13 weight/volume to methanol used.

Preferably, the solution is maintained, while stirring, at a temperature of about −15° C., for about 1 hour, to obtain Form C.

Tegaserod maleate Form C may then be recovered by any method known in art.

Pharmaceutical compositions containing crystalline tegaserod maleate may optionally contain a mixture of other form(s) of tegaserod maleate. In addition to the active ingredient(s), the pharmaceutical formulations may contain one or more excipients. Excipients are added to the formulation for a variety of purposes.

Pharmaceutical compositions may be prepared as medicaments to be administered orally, parenterally, rectally, transdermally, bucally, or nasally. Suitable forms for oral administration include tablets, compressed or coated pills, dragees, sachets, hard or gelatin capsules, sub-lingual tablets, syrups and suspensions. Suitable forms of parenteral administration include an aqueous or non-aqueous solution or emulsion, while for rectal administration suitable forms for administration include suppositories with hydrophilic or hydrophobic vehicle. For topical administration the invention provides suitable transdermal delivery systems known in the art, and for nasal delivery there are provided suitable aerosol delivery systems known in the art.

Selection of excipients and the amounts to use may be readily determined by the formulation scientist based upon experience and consideration of standard procedures and reference works in the field. For example, diluents increase the bulk of a solid pharmaceutical composition, and may make a pharmaceutical dosage form containing the composition easier for the patient and care giver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®) and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions, the active ingredient and any other solid excipients are dissolved or suspended in a liquid carrier such as water, vegetable oil, alcohol, polyethylene glycol, propylene glycol or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol.

Liquid pharmaceutical compositions of the present invention may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum.

Sweetening agents such as sorbitol, saccharin, sodium saccharin, sucrose, aspartame, fructose, mannitol and invert sugar may be added to improve the taste.

Preservatives and chelating agents such as alcohol, sodium benzoate, butylated hydroxy toluene, butylated hydroxyanisole and ethylenediamine tetraacetic acid may be added at levels safe for ingestion to improve storage stability.

According to the present invention, a liquid composition may also contain a buffer such as gluconic acid, lactic acid, citric acid or acetic acid, sodium gluconate, sodium lactate, sodium citrate or sodium acetate.

Selection of excipients and the amounts used may be readily determined by the formulation scientist based upon experience and consideration of standard procedures and reference works in the field.

The solid compositions of the present invention include powders, granulates, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions and elixirs.

The dosage form of the present invention may be a capsule containing the composition, preferably a powdered or granulated solid composition of the invention, within either a hard or soft shell. The shell may be made from gelatin and optionally contain a plasticizer such as glycerin and sorbitol, and an opacifying agent or colorant.

The active ingredient and excipients may be formulated into compositions and dosage forms according to methods known in the art.

A composition for tableting or capsule filling may be prepared by wet granulation. In wet granulation, some or all of the active ingredients and excipients in powder form are blended and then further mixed in the presence of a liquid, typically water, that causes the powders to clump into granules. The granulate is screened and/or milled, dried and then screened and/or milled to the desired particle size. The granulate may then be tabletted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling form of the present invention may comprise any of the aforementioned blends and granulates described for tableting. In capsule filling, however, the blends and granulates are not subjected to the final tableting step.

The pharmaceutical compositions of the present invention, used to treat irritable bowel syndrome in a mammal such as a human, are preferably in the form of a coated tablet, and are administered on an empty stomach twice a day, for a period of about 4 to about 6 weeks. Additional administration may occur if the patient responds positively to the treatment.

Having thus described the invention with reference to particular preferred embodiments and illustrative examples, those in the art may appreciate modifications to the invention as described and illustrated that do not depart from the spirit and scope of the invention as disclosed in the specification. The following examples are set forth to aid in understanding the invention but are not intended to, and should not be construed to, limit its scope in any way. The examples do not include detailed descriptions of conventional methods. Such methods are well known to those of ordinary skill in the art and are described in numerous publications. Polymorphism in Pharmaceutical Solids, Drugs and the Pharmaceutical Sciences, Volume 95 may be used as a guidance.

### EXAMPLES

Instrumentation

Spray drying was performed on a Buchi Mini Spray dryer B-290 with an evaporating capacity of 1 L/hr for water and higher for organic solvents. The maximum temperature input was 220° C., the air flow was at a maximum of 35 m²/hr, and the spray gas was compressed air or nitrogen at 200-800 L/hr and 5-8 bar. The nozzle diameter was 0.7 mm (standard), and the nozzle cap was 1.4 mm and 1.5 mm.

X-Ray powder diffraction (XRD) data is obtained using a SCINTAG powder X-Ray diffractometer model X'TRA equipped with a solid state detector. Copper radiation of 1.5418 Å is used. A round aluminum sample holder with zero background is used. All peak positions are within ±0.2 degrees two theta.

Sonicator: Sonics Vibra-cell, amplitude: 35, power 1500 W.

### Example 1

### Preparation of Crystalline Tegaserod Maleate Form A and B3

Tegaserod maleate (5 g) was dissolved in water (7 ml) and ethanol absolute (28 ml) at reflux temperature. The obtained solution was pumped into the spray dryer and contacted with nitrogen gas. The inlet temperature of the nitrogen gas was 100° C. The evaporated solvents and nitrogen exited the spray dryer at 67-68° C. The product was analyzed by XRD and found to be mixture of Forms B3 and A.

### Example 2

### Preparation of Crystalline Tegaserod Maleate Form A and B2

Tegaserod maleate (5 g) was dissolved in water (26 ml) and ethanol absolute (104 ml) at about 70° C. The obtained solution was pumped into the spray dryer and contacted with nitrogen gas. The inlet temperature of the nitrogen gas was 50° C. The evaporated solvents and nitrogen exited the spray dryer at 33-34° C. The product was analyzed by XRD and found to be mixture of Forms B2 and A.

### Example 3

### Preparation of Crystalline Tegaserod Maleate Form A

Tegaserod maleate (9 g) was dissolved in N-methyl-2-pyrrolidine (90 ml) at room temperature. The obtained solution was pumped into the spray dryer in two portions. Example 3A. Preparation of crystalline tegaserod maleate Form A The first portion of the obtained solution was pumped into the spray dryer and contacted with nitrogen gas. The inlet temperature of the nitrogen gas was 100° C. The evaporated solvent and nitrogen exited the spray dryer at 68° C. The product was analyzed by XRD and found to be Form A.

### Example 3B

### Preparation of Crystalline Tegaserod Maleate Form A

The second portion of the obtained solution was pumped into the spray dryer and contacted with nitrogen gas. The inlet temperature of the nitrogen gas was 150° C. The evaporated solvent and nitrogen exited the spray dryer at 98° C. The product was analyzed by XRD and found to be Form A.

### Example 4

### Preparation of Crystalline Tegaserod Maleate Form A

Tegaserod maleate (10 g) was dissolved in N,N-dimethyl formamide (250 ml) at room temperature. The obtained solution was pumped into the spray dryer in three portions.

### Example 4A

### Preparation of Crystalline Tegaserod Maleate Form A

The first portion of the obtained solution was pumped into a spray dryer and contacted with nitrogen gas. The inlet temperature of the nitrogen gas was 50° C. The evaporated solvent and nitrogen left the spray dryer at a temperature of 38-40° C. The product was analyzed by XRD and found to be Form A.

### Example 4B

### Preparation of Crystalline Tegaserod Maleate Form A

The second portion of the obtained solution was pumped into a spray dryer and contacted with nitrogen gas. The inlet temperature of the nitrogen gas was 100° C. The evaporated solvent and nitrogen left the spray dryer at a temperature of 68-70° C. The product was analyzed by XRD and found to be Form A.

### Example 4C

### Preparation of Crystalline Tegaserod Maleate Form A

The third portion of the obtained solution was pumped into a spray dryer and contacted with nitrogen gas. The inlet temperature of the nitrogen gas was 150° C. The evaporated solvent and nitrogen left the spray dryer at a temperature of 97-100° C. The product was analyzed by XRD and found to be Form A.

### Example 5

### Preparation of Crystalline Tegaserod Maleate Form A

Tegaserod maleate (5 g) was dissolved in N-methyl-2-pyrrolidine (12.5 ml) at room temperature and the solution was pumped into the spray dryer and contacted with nitrogen gas. The inlet temperature of the nitrogen gas was 100° C. The evaporated solvent and nitrogen left the spray dryer at a temperature of 66-69° C. The product was analyzed by XRD and found to be Form A.

### Example 6

### Preparation of Crystalline Tegaserod Maleate Form A

Tegaserod maleate (5 g) was dissolved in N-methyl-2-pyrrolidine (500 ml) at room temperature and the solution was pumped into the spray dryer and contacted with nitrogen gas. The inlet temperature of the nitrogen gas was 150° C. The evaporated solvent and nitrogen left the spray dryer at a temperature of 96-97° C. The product was analyzed by XRD and found to be Form A.

### Example 7

### Preparation of Crystalline Tegaserod Maleate Form A

Tegaserod maleate (10 g) was dissolved in acetone (256 ml) and water (64 ml) at room temperature and the solution was pumped into the spay dryer in three portions.

### Example 7A

### Preparation of Crystalline Tegaserod Maleate Form A

The first portion of the obtained solution was pumped into the spray dryer and contacted with nitrogen gas. The inlet temperature of the nitrogen gas was 50° C. The evaporated solvent and nitrogen left the spray dryer at a temperature of 37-39° C. The product was analyzed by XRD and found to be Form A.

### Example 7B

### Preparation of Crystalline Tegaserod Maleate Form A

The second portion of the obtained solution was pumped into the spray dryer and contacted with nitrogen gas. The inlet temperature of the nitrogen gas was 100° C. The evaporated solvent and nitrogen left the spray dryer at a temperature of 69-72° C. The product was analyzed by XRD and found to be Form A.

### Example 7C

### Preparation of Crystalline Tegaserod Maleate Form A

The third portion of the obtained solution was pumped into the spray dryer and contacted with nitrogen gas. The inlet temperature of the nitrogen gas was 150° C. The evaporated solvent and nitrogen left the spray dryer at a temperature of 95-101° C. The product was analyzed by XRD and found to be Form A.

### Example 8

### Preparation of Crystalline Tegaserod Maleate Form A

Tegaserod maleate (4.2 g) was dissolved in N-methyl-2-pyrrolidine (21 ml) and methanol (21 ml) at room temperature and the solution was pumped into the spray dryer and contacted with nitrogen gas. The inlet temperature of the nitrogen gas was 100° C. The evaporated solvent and nitrogen left the spray dryer at a temperature of 69-73° C. The product was analyzed by XRD and found to be Form A.

### Example 9

### Preparation of Crystalline Tegaserod Maleate Form A

Tegaserod maleate (3.5 g) was dissolved in N-methyl-2-pyrrolidine (35 ml) and methanol (35ml) at room temperature and the solution was pumped into the spray dryer and contacted with nitrogen gas. The inlet temperature of the nitrogen gas was 100° C. The evaporated solvent and nitrogen left the spray dryer at a temperature of 70-72° C. The product was analyzed by XRD and found to be Form A.

### Example 10

### Preparation of Crystalline Tegaserod Maleate Form A

Tegaserod maleate (8 g) was dissolved in N-methyl-2-pyrrolidine (30 ml) and methanol (90 ml) at room temperature and the solution was pumped into the spray dryer in two portions.

### Example 10A

### Preparation of Crystalline Tegaserod Maleate Form A

The first portion of the obtained solution was pumped into a spray dryer and contacted with nitrogen gas. The inlet temperature of the nitrogen gas was 100° C. The evaporated solvent and nitrogen left the spray dryer at a temperature of 66-72° C. The product was analyzed by XRD and found to be Form A.

### Example 10B

### Preparation of Crystalline Tegaserod Maleate Form A

The second portion of the obtained solution was pumped into a spray dryer and contacted with nitrogen gas. The inlet temperature of the nitrogen gas was 150° C. The evaporated solvent and nitrogen left the spray dryer at a temperature of 91-98° C. The product was analyzed by XRD and found to be Form A.

### Example 11

### Preparation of Tegaserod Maleate Form A from Tegaserod Hemi-Maleate Hemihydrate Without Solvent

1 g of Tegaserod Hemi-maleate hemihydrate and 0.16 g maleic acid were grounded together in a mortar for 10 minutes. The product was analyzed by XRD and found to be Form A.

### Example 12

### Preparation of Tegaserod Maleate Form A from Tegaserod Hemi-Maleate Hemihydrate in the Presence of a Sonicator

A mixture of 3 g of Tegaserod Hemi-maleate hemihydrate in 40 mL ethyl acetate at room temperature was treated with a sonicator (set up at an amplitude of 35, 1500 watt) and a solution of 0.47 g of maleic acid in ethyl acetate/water (90:10) was added, and the slurry was stirred for 40. The resulting solid was filtered off and washed with the same solution. After drying on vacuum oven at 45° C. for 15 hours, 1.41 g of tegaserod maleate were obtained. The product was analyzed by XRD and found to be Form A.

### Example 13

### Preparation of Tegaserod Maleate Form A from Tegaserod Hemi-Maleate Hemihydrate

A slurry of Tegaserod Hemi-maleate hemihydrate (2 g) in the appropriate solvent was heated to 60-65° C. and a solution of 0.77 g maleic acid in 10 ml of the same solvent was added. The mixture was stirred 2 hours at the same temperature followed by cooling to room temperature and stirring for 3 hours. The resulting solid was filtered off and washed with the same solvent. After drying on vacuum oven at 45° C. for 15 hours the product was analyzed by XRD and found to be Form A.

| Solvent | Total Volume (ml/g) |
|---|---|
| Water | 45 |
| Acetonitrile | 40 |
| n-butanol | 40 |
| Sec-butanol | 40 |
| MIBK | 40 |

### Example 14

### Preparation of Tegaserod Maleate Form A from Tegaserod Hemi-Maleate Hemihydrate

A slurry of Tegaserod Hemi-maleate hemihydrate (2 g) in the appropriate solvent was heated to 60-65° C. and a solution of 0.77 g maleic acid in 10 ml of the same solvent was added. The mixture was stirred 2 hours at the same temperature followed by cooling to room temperature and stirring for overnight. The resulting solid was filtered off and washed with the same solvent. After drying on vacuum oven at 45° C. for 15 hours the product was analyzed by XRD and found to be Form A.

| Solvent | Total Volume (ml/g) |
|---|---|
| Toluene | 40 |
| Heptane | 40 |
| MEK | 40 |

### Example 15

### Preparation of Tegaserod Maleate Form A from Tegaserod Hemi-Maleate Hemihydrate

A slurry of Tegaserod Hemi-maleate hemihydrate (2 g) in the appropriate solvent was kept at room temperature and 0.32 g of maleic acid were added. The mixture was stirred at room temperature overnight. The resulting solid was filtered off and washed with the same solvent. After drying on vacuum oven at 45° C. for 15 hours the product was analyzed by XRD and found to be Form A.

| Solvent | Total Volume (ml/g) |
|---|---|
| Toluene | 5 |
| Heptane | 5 |
| DIPE | 5 |
| 2-methyl-THF | 5 |

### Example 16

### Preparation of Tegaserod Maleate Form B from Tegaserod Hemi-Maleate Hemihydrate

A slurry of Tegaserod Hemi-maleate hemihydrate (2 g) in n-propanol (42.5 ml/g) was heated to 60-65° C. and a solution of 0.32 g maleic acid in 5 ml of the same solvent was added. The mixture was stirred 2 hours at the same temperature followed by cooling to room temperature and stirring for 3 hours. The resulting solid was filtered off and washed with the same solvent. After drying on vacuum oven at 45° C. for 15 hours the product was analyzed by XRD and found to be Form B.

### Example 17

### Preparation of Tegaserod Maleate Form B from Tegaserod Hemi-Maleate Hemihydrate

To a slurry of Tegaserod Hemi-maleate hemihydrate (40 g) in 200 ml n-propanol were added 6 g of maleic acid. The mixture was heated to 60-65° C. and stirred 3 hours at the same temperature followed by cooling to room temperature and stirring for 2 hours. The resulting solid was filtered off and washed with 40 ml of the same solvent. After drying on vacuum oven at 45° C. and the product was analyzed by XRD and found to be Form B.

### Example 18

### Preparation of Tegaserod Maleate Form B from Tegaserod Maleate Form B2 in a Mixture of Ethyl Acetate/n-Propanol

15 gr of TGS Maleate (form B2), 270 ml of Ethyl Acetate and 270 ml of n-Propanol (ratio 1:1) were added to a stirred reactor. The reactor jacket was heated to 100 deg. and the mixture was stirred for 0.5 hr. The mixture was cooled to 10 deg and then stirred for 0.5 hr. The mixture was filtrated under vacuum and the solids were washed twice with 30 ml of n-Propanol. The wet product was dried in a vacuum oven to obtain 14.4 gr of dry product. (Yield=96%). The dry product was identified by XRD as TGS Ma form B.

### Example 19

### Preparation of Tegaserod Maleate Form B from Tegaserod Maleate Form B2 in a Mixture of Ethyl Acetate/n-Propanol

15 gr of TGS Maleate (form B2), 135 ml of Ethyl Acetate and 405 ml of n-Propanol (ratio 1:3) were added to a stirred reactor. The reactor jacket was heated to 100 deg. and the mixture was stirred for 0.5 hr. The mixture was cooled to 10 deg and then stirred for 0.5 hr. The mixture was filtrated under vacuum and the solids were washed twice with 30 ml of n-Propanol. The wet product was dried in a vacuum oven to obtain 14.9 gr of dry product. (Yield=99%). The dry product was identified by XRD as TGS Ma form B.

### Example 20

### Preparation of Tegaserod Maleate Form B by Crystallization

A slurry of tegaserod maleate (2.06 g) in the appropriate solvent (5 mL) was heated to reflux, and then additional solvent was added until complete dissolution. After the compound was dissolved, the oil bath was removed and the solution was cooled to room temperature and stirred for an additional 7 hours. The solid was filtrated and washed with 5 mL of the same solvent and dried in a vacuum oven at 40° C. for 16 hours. The product was analyzed by XRD and found to be form B.

| Solvent | Volume (ml) |
|---|---|
| Ethyl acetate/n-propanol 1:1 | 140 |
| Ethyl acetate/n-propanol 1:3 | 70 |

### Example 21

### Preparation of Tegaserod Maleate Form B1 from Tegaserod Hemi-Maleate Hemihydrate

A slurry of Tegaserod Hemi-maleate hemihydrate (2 g) in 80 ml isopropyl alcohol was heated to 60-65° C. and a solution of 0.32 g maleic acid in 5 ml of isopropyl alcohol was added. The mixture was stirred for 2 hours at the same temperature followed by cooling to room temperature and stirring for 3 hours. The resulting solid was filtered off and washed with ethanol. After drying on vacuum oven at 45° C. for 15 hours the product was analyzed by XRD and found to be Form B1.

### Example 22

### Preparation of Tegaserod Maleate Form B2 from Tegaserod Hemi-Maleate Hemihydrate

A slurry of Tegaserod Hemi-maleate hemihydrate (2 g) in the appropriate solvent was heated to 60-65° C. and a solution of 0.32 g maleic acid in 5 ml of the same solvent was added. The mixture was stirred 2 hours at the same temperature followed by cooling to room temperature and stirring for 3 hours. The resulting solid was filtered off and washed with the same solvent. After drying on vacuum oven at 45° C. for 15 hours the product was analyzed by XRD and found to be Form B2.

| Solvent | Total Volume (ml/g) |
|---|---|
| EtOH/water (1:1) | 42.5 |
| EtOH/water (8:2) | 42.5 |
| EtOH/water (7:3) | 42.5 |

### Example 23

### Preparation of Tegaserod Maleate Form B3 from Tegaserod Hemi-Maleate Hemihydrate

A slurry of Tegaserod Hemi-maleate hemihydrate (2 g) in 70 ml ethanol was heated to 60-65° C. and a solution of 0.77 g maleic acid in 10 ml of the same solvent was added. The mixture was stirred 2 hours at the same temperature followed by cooling to room temperature and stirring for 3 hours. The resulting solid was filtered off and washed with ethanol. After drying on vacuum oven at 45° C. for 15 hours the product was analyzed by XRD and found to be Form B3.

### Example 24

### Preparation of Tegaserod Maleate Form M from Tegaserod Hemi-Maleate Hemihydrate

A slurry of Tegaserod Hemi-maleate hemihydrate (2 g) in 70 ml acetone was heated to reflux and a solution of 0.77 g maleic acid in 10 ml of the same solvent was added. The mixture was stirred 2 hours at the same temperature followed by cooling to room temperature and stirring for 3 hours. The resulting solid was filtered off and washed with acetone. After drying on vacuum oven at 45° C. for 15 hours the product was analyzed by XRD and found to be Form M.

### Example 25

### Preparation of Tegaserod Maleate Form C from Tegaserod Hemi-Maleate Hemihydrate

A slurry of Tegaserod Hemi-maleate hemihydrate (2 g) in 70 ml methanol was heated to reflux and a solution of 0.32 g maleic acid in 10 ml of the same solvent was added. The mixture was stirred 2 hours at the same temperature followed by cooling to room temperature and stirring for 3 hours. After filtration and drying on vacuum oven at 45° C. for 15 hours the product was analyzed by XRD and found to be Form C.

### Example 26

### Preparation of Tegaserod Maleate Form C from Tegaserod Hemi-Maleate Hemihydrate

To a slurry of Tegaserod Hemi-maleate hemihydrate (2 g) in 40 ml methanol at room temperature was added a solution of 0.31 g maleic acid in 5 ml of acetone was added. The mixture was stirred 3 hours at room temperature, and the resulting solid was filtered off and washed with acetone. After drying on vacuum oven at 45° C. for 15 hours the product was analyzed by XRD and found to be Form C.

### Example 27

### Preparation of Tegaserod Maleate Form M

A slurry of tegaserod maleate (2.06 g) in 20 ml ethyl acetate/acetonitrile 1:3, was heated to reflux, and then, 170 ml of ethyl acetate/acetonitrile 1:3 were added until the solid completely dissolved. The hot solution was filtered and stirred at room temperature for an additional 1.5 hours. The precipitate was filtered and washed with 10 mL of ethyl acetate/acetonitrile 1:3 and dried in a vacuum oven at 40° C. overnight.

### Example 28

### Preparation of Tegaserod Maleate Form M

A slurry of tegaserod maleate (2.06 g) in 20 ml ethyl acetate/acetonitrile 1:3, was heated to reflux, and then, 170 ml of ethyl acetate/acetonitrile 1:3 were added until the solid completely dissolved. The hot solution was filtered and stirred at room temperature for an additional 1.5 hours. The precipitate was filtered and washed with 10 mL of ethyl acetate/acetonitrile 1:3. The wet material was analyzed by XRD and found to be Form M.

### Example 29a

### Preparation of Tegaserod Maleate Form C

30 g of tegaserod hemi-maleate hemihydrate, 4.8 g maleic acid and 450 ml of methanol were added to a stirred reactor. The reactor jacket was heated to 80° C. while stirring. The mixture was then stirred for an additional 3 hours. 150 ml of methanol were then added to obtain a clear solution.

150 ml of methanol were charged to another reactor and cooled to −15° C. The hot mixture was added dropwise to the cold methanol over a period of 45 minutes. Precipitation occurred during the addition. After the addition was complete, the mixture was stirred at −15° C. for an additional 1 hour. The mixture was then filtered under vacuum and the solids were washed with 60 ml of methanol.

34.2 g of wet product were obtained and identified as tegaserod maleate form B2 by XRD.

32.5 g of the wet product was dried in a vacuum oven to obtain 27.5 g of dry product (Yield=85. 1%). The dry product was identified by XRD as tegaserod maleate form C.

### Example 29b

### Preparation of Form Z

The tegaserod maleate form C obtained in Example 29a was dried in a conventional oven at 120° C. for two hours. The dried product was identified by XRD as Form Z.

What is claimed is:

1. A process for preparing crystalline tegaserod maleate characterized by an X-ray diffraction pattern with peaks at about 15.7, 16.9, 17.2, 24.1, 24.6 and 25.2±0.2 degrees two theta comprising:
a. combining a slurry of tegaserod hemi-maleate hemihydrate in n-propanol, with maleic acid to obtain a mixture;
b. maintaining the mixture; and
c. recovering the crystal form.

2. The process of claim 1, wherein before combining the slurry with maleic acid, the slurry is heated to a temperature of from about room temperature to about 70° C.

3. The process of claim 2, wherein the temperature is from about 60° C. to about 65° C.

4. The process of claim 19, wherein the mixture is cooled to room temperature.

5. The process of claim 1, wherein the maleic acid is added as a solution with the same solvent used to form the slurry.

6. A process for preparing crystalline tegaserod maleate characterized by an X-ray diffraction pattern with peaks at about 15.7, 16.9, 17.2, 24.1, 24.6 and 25.2±0.2 degrees two theta comprising:
a. combining tegaserod maleate crystalline form characterized by an X-ray diffraction pattern with peaks at about 8.7, 15.6, 16.0, 22.2 and 25.3±0.2 degrees two theta with ethyl acetate and n-propanol to obtain a mixture;
b. heating the mixture to a temperature of about 100° C. to reflux;
c. cooling the mixture to about room temperature or less; and
d. recovering the crystal form.

7. The process of claim 6, wherein the mixture is heated to a temperature of about 100° C.

8. The process of claim 6, wherein the mixture is cooled to a temperature of about 10° C.

9. A process for crystallizing tegaserod maleate characterized by an X-ray diffraction pattern with peaks at about 15.7, 16.9, 17.2, 24.1, 24.6 and 25.2±0.2 degrees two theta from a solution of tegaserod maleate, ethyl acetate and n-propanol.

10. The process of claim 9, wherein the ethyl acetate used is in a ratio of about 1:1 to about 1:3 by volume to n-propanol used.

11. A process for preparing crystalline tegaserod maleate characterized by an X-ray diffraction pattern with peaks at about 5.4, 6.0, 6.6 and 10.8±0.2 degrees two theta, comprising spray drying a solution of tegaserod maleate in a solvent selected from the group consisting of: amines, amides, ketones and mixtures thereof with C₁-C₈ alcohols or water.

12. The process of claim 11, wherein the solvent is selected from the group consisting of: N-methyl-2-pyrrolidine or mixture thereof with methanol, N,N-dimethylformamide, and a mixture of acetone and water.

13. The process of claim 11, wherein the solution is spray-dried at an inlet temperature of from about 30° C. to about 200° C.

14. The process of claim 13, wherein the solution is spray-dried at an inlet temperature of from about 50° C. to about 200° C.

15. The process of claim 14, wherein the solution is spray-dried at an inlet temperature of from about 50° C. to about 150° C.

16. A process for preparing crystalline tegaserod maleate characterized by an X-ray diffraction pattern with peaks at about 5.4, 6.0, 6.6 and 10.8±0.2 degrees two theta, comprising grinding a mixture of tegaserod hemi-maleate hemihydrate with maleic acid.

17. The process of claim 16, wherein, the tegaserod hemi-maleate hemihydrate is present in a ratio of about 1:1 weight /volume of maleic acid.

18. A process for preparing crystalline tegaserod maleate characterized by an X-ray diffraction pattern with peaks at about 5.4, 6.0, 6.6 and 10.8±0.2 degrees two theta, comprising:
a. combining a slurry of tegaserod hemi-maleate hemihydrate in a solvent selected from the group consisting of: ethyl acetate, diisopropyl ether [DIPE], 2-methyl-THF, water, acetonitrile, n-butanol, sec-butanol, methyl isobutyl ketone, toluene, heptane, MEK or a mixture thereof, with maleic acid to obtain a mixture;
b. maintaining the mixture; and
c. recovering the crystal form.

19. The process of claim 18, wherein before combining the slurry with maleic acid, the slurry is heated to a temperature of from about room temperature to about 70° C.

20. The process of claim 19, wherein the temperature is from about 60° C. to about 65° C.

21. The process of claim 19, wherein the mixture is cooled to room temperature.

22. The process of claim 18, wherein the maleic acid is added as a solution with the same solvent used to form the slurry.

23. The process of claim 18, wherein the mixture is treated with an ultrasound probe (sonicator).

24. A process for preparing crystalline tegaserod maleate characterized by an X-ray diffraction pattern with peaks at about 6.6, 7.9, 8.9, 19.7, 21.8, 23.0, 23.9, 25.3 and 27.2 degrees two-theta, ±0.2 degrees two-theta comprising:
a. combining a slurry of tegaserod hemi-maleate hemihydrate in methanol, with maleic acid to obtain a mixture;
b. maintaining the mixture; and
c. recovering the obtained crystal form.

25. The process of claim 24, wherein before combining the slurry with maleic acid, the slurry is heated to a temperature of from about room temperature to about 70° C.

26. The process of claim 25, wherein the temperature is from about 60° C. to about 65° C.

27. The process of claim 25, wherein the mixture is cooled to room temperature.

28. The process of claim 24, wherein the maleic acid is added as a solution with acetone.

29. A process for crystallizing tegaserod maleate characterized by an X-ray diffraction pattern with peaks at about 6.6, 7.9, 8.9, 19.7, 21.8, 23.0, 23.9, 25.3 and 27.2±0.2 degrees two-theta from a solution of tegaserod hemi-maleate hemihydrate, maleic acid and methanol.

30. The process of claim 29, wherein maleic acid used is in a ratio of about 1:10 to about 1:13 weight/volume to methanol used.

31. A crystalline form of tegaserod maleate characterized by X-ray powder diffraction peaks at about 6.6, 7.9, 8.9, 19.7 and 27.2±0.2 degrees two-theta, wherein the crystalline form is substantially free of a peak at about 10.3±0.2 degrees two theta.

32. The crystalline form of claim 31, further characterized by X-ray powder diffraction peaks at about 21.8, 23.0, 23.9 and 25.3±0.2 degrees two-theta.

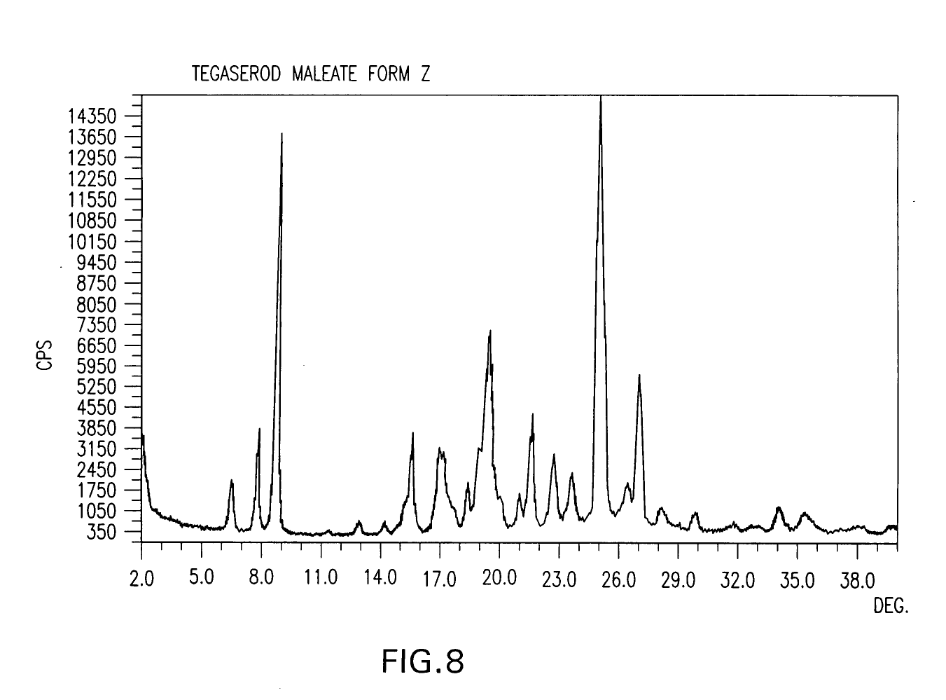

33. The crystalline form of claim 31, wherein the crystalline form has an X ray diffraction pattern as substantially depicted in FIG. 8.

34. The crystalline form of claim 31, wherein the peak at about 10.3±0.2 degrees two theta is absent.

35. The crystalline form of claim 31, containing less than about 5% of any other crystalline form of tegaserod maleate by weight.

36. The crystalline form of claim 35, containing less than about 1% of any other crystalline form of tegaserod maleate by weight.

37. The crystalline form of claim 36, free of detectable peaks at about 7.0, 10.3, 13.7, 20.7 and 23.2±0.2 degrees two theta.

38. The crystalline form of claim 31, which is anhydrous.

39. The crystalline form of claim 31, which has a weight loss of about 0.1% by weight at the range of about 25° C. to about 200° C., as measured by TGA.

40. A pharmaceutical composition comprising the crystalline form of claim 31, and a pharmaceutically acceptable excipient

41. A process for preparing crystalline tegaserod maleate characterized by an X-ray diffraction pattern having peaks at about 10.3, 16.1, 16.5, 17.1, 20.3, 22.0 and 25.3±0.2 degrees two theta comprising:
a. combining a slurry of tegaserod hemi-maleate hemihydrate in iso-propyl alcohol (IPA), with maleic acid to obtain a mixture;
b. maintaining the mixture; and
c. recovering the crystal form.

42. A process for preparing crystalline tegaserod maleate characterized by an X-ray diffraction pattern having peaks at about 8.7, 15.6, 16.0, 22.2 and 25.3±0.2 degrees two theta comprising:
a. combining a slurry of tegaserod hemi-maleate hemihydrate in ethanol/water, with maleic acid to obtain a mixture;
b. maintaining the mixture; and
c. recovering the crystal form.

43. A process for preparing crystalline tegaserod maleate characterized by an X-ray diffraction pattern having peaks at about 15.6, 16.0, 22.5, 25.5 and 29.3±0.2 degrees two theta comprising:
a. combining a slurry of tegaserod hemi-maleate hemihydrate in ethanol, with maleic acid to obtain a mixture;
b. maintaining the mixture; and
c. recovering the crystal form.

44. A process for preparing tegaserod maleate Form M comprising:
a. combining a slurry of tegaserod hemi-maleate hemihydrate in acetone, with maleic acid to obtain a mixture;
b. maintaining the mixture; and
c. recovering the crystal form.

45. A process for crystallizing Form M from a solution of tegaserod maleate and a mixture of ethyl acetate and acetonitrile.
